(19) United States
(12) Patent Application Publication
(10) Pub. No.: US 2006/0172302 A1
(43) Pub. Date: Aug. 3, 2006

(54) Assay method

(76) Inventors: Arne Hermansen, Fredrik A Dahls vie 20 (NO); Sonja Klemsdal, Fredrik A Dahls vie 20 (NO); Ragnhild Naerstad, Fredrik A Dahls vie 20 (NO); Leslie Wanner, Fredrik A Dahls vie 20 (NO); Grete Lund, Fredrik A Dahls vie 20 (NO)

Correspondence Address: SUGHRUE MION, PLLC, 2100 PENNSYLVANIA AVENUE, N.W., SUITE 800, WASHINGTON, DC, 20037, US

(21) Appl. No.: 10/533,166

(22) PCT Filed: Oct. 31, 2003
(86) PCT No.: PCT/GB03/04712

§ 371 Date: Jan. 17, 2006

(30) Foreign Application Priority Data
Nov. 1, 2002 (GB) 0225550.3
Nov. 1, 2002 (GB) 0225551.1

(51) Int. Cl. C12Q 1/68; C07H 21/04

(52) U.S. Cl. 435/6; 536/23.7

(57) ABSTRACT

The invention provides an assay method for detecting fungal infection of soil or vegetables by pathogenic fungal species, in particular M. acerina, F. carotae and Pythium species, said method comprising: obtaining a sample of soil or vegetable; treating said sample to lyse fungal cells therein; using an oligonucleotide primer pair, effecting a polymerase chain reaction on DNA released by lysis of the fungal cells; and detecting DNA fragments generated by said polymerase chain reaction; wherein said primer pair comprises an 18- to 24-mer having the ability to hybridize to one of the oligonucleotide sequences of formulae (Ia), (Ib), (IIa), (IIb), (IIIa), (IIIb), (IVa), (IVb), (Va), (Vb), (VIa), (VIb), (VIIa), (VIIb), (VIIIa), (VIIIb), (IXa), (IXb), (Xa), (Xb), (XIa), (XIb), (XIIa), (XIIb), (XIIIa), (XIIIb), (XIVa) and (XIVb).

This invention relates to an assay method for detecting fungal infection of fields and vegetables, and to compounds, kits and microarrays for use in such assays.

Almost one third of the carrot crop is lost worldwide due to pests and diseases.

While chemical treatment of carrot growing fields and of harvested carrots can be used to reduce the loss in the carrot crop, this is expensive and means that the carrots can not be sold as “organic”.

There is thus a pressing need for a diagnostic method with the use of which loss in crop yield may be reduced.

Root vegetables like carrots are particularly susceptible to pathogens present in the soil in which they are grown and especially to fungal infection. Such fungal infection can cause damage to the carrots while still in the ground or the damage may occur later during post-harvest storage.

One especially damaging fungal infection of carrots is called cavity spot and is caused by fungi of the species Pythium, especially P. viola and P. sulcatum. This infection damages the surface of the carrot root while it is still in the ground and renders the carrots essentially worthless.

Another especially damaging fungal infection of carrots is called liquorice rot and is caused by the fungus Mycocentrospora acerina. A still further especially damaging fungal infection of carrots is called crater rot and is caused by the fungus Fibularhizoctonia carotae. Both of these infections develop during post-harvest storage and also render the carrots essentially worthless.

Fungal infection of carrot growing fields may, as mentioned above, be treated by spraying the fields with antifungal agents, e.g. metalaksyl. Alternatively the infected fields may be used for other crops not sensitive to fungal infection by Pythium species, M. acerina or F. carotae until the infection has disappeared. However waiting for the infection to clear is a long and uncertain business as the fungus may have other host species available and as viable fungal spores can remain dormant in the soil for years.

We have now found that soil from fields in which carrots might be grown may be analysed to determine whether the fields are infected with Pythium, M. acerina or F. carotae, thus enabling the grower to decide whether to spray with an antifungal agent or to avoid sowing such fields with carrots until a later season when the infection has disappeared. Likewise tissue or surface soil from symptom-free carrots may be tested, e.g. before or shortly after harvesting, to determine whether treatment with a fungicide to prolong storage life is necessary or to determine whether the carrots should be used (e.g. sold, cooked, bottled, canned etc) promptly rather than stored for prolonged periods.

Thus viewed from one aspect the invention provides an assay method for detecting fungal infection of soil or vegetables by pathogenic fungal species, in particular M. acerina, F. carotae and Pythium species, said method comprising:
obtaining a sample of soil or vegetable; treating said sample to lyse fungal cells therein; using an oligonucleotide primer pair, effecting a polymerase chain reaction on DNA released by lysis of the fungal cells; and detecting DNA fragments generated by said polymerase chain reaction;

wherein said primer pair comprises an 18- to 24-mer having the ability to hybridize to one of the oligonucleotide sequences of formulae Ia (SEQ ID NO:1), Ib (SEQ ID NO:2), IIa (SEQ ID NO:3), IIb (SEQ ID NO:4), IIIa (SEQ ID NO:5), IIIb (SEQ ID NO:6), IVa (SEQ ID NO:7), IVb (SEQ ID NO:8), Va (SEQ ID NO:9), Vb (SEQ ID NO:10), VIa (SEQ ID NO:11), VIb (SEQ ID NO:12), VIIa (SEQ ID NO:13), VIIb (SEQ ID NO:14), VIIIa (SEQ ID NO:15), VIIIb (SEQ ID NO:16), IXa (SEQ ID NO:17), IXb (SEQ ID NO:18), Xa (SEQ ID NO:19), Xb (SEQ ID NO:20), XIa (SEQ ID NO:21), XIb (SEQ ID NO:22), XIIa (SEQ ID NO:23), XIIb (SEQ ID NO:24), XIIIa (SEQ ID NO:25), XIIIb (SEQ ID NO:26), XIVa (SEQ ID NO:27) and XIVb (SEQ ID NO:28):

| | |
|---|---|
| 5′ - TCA CTT GTG GGG TAA AGA AGA - 3′ | (Ia) |
| 5′ - AGA CCA CAA TAA AGC GGC - 3′ | (Ib) |
| 5′ - AGT CCC GCA CAC ACA CAT - 3′ | (IIa) |
| 5′ - ACT TCT CTC TTT GGG GAG TGG - 3′ | (IIb) |
| 5′ - TTC GTT CAG CCT CTG CAT - 3′ | (IIIa) |
| 5′ - TCG TTT CGG CTA TGA ATA CAG - 3′ | (IIIb) |
| 5′ - ACA AAT ATA CCA ACC ACA GCG - 3′ | (IVa) |
| 5′ - TTT GTA CTT GTG CAA TTG GC - 3′ | (IVb) |
| 5′ - AAC GAA TAT ACC AAC CGC TG - 3′ | (Va) |
| 5′ - TCA TCT ATT TGT GCA CTT CTT TTT - 3′ | (Vb) |
| 5′ - TCT TCT TTA CCC CAC AAG TGA - 3′ | (VIa) |
| 5′ - GCC GCT TTA TTG TGG TCT - 3′ | (VIb) |
| 5′ - ATG TGT GTG TGC GGG ACT - 3′ | (VIIa) |
| 5′ - CCA CTC CCC AAA GAG AGA AGT - 3′ | (VIIb) |
| 5′ - ATG CAG AGG CTG AAC GAA - 3′ | (VIIIa) |
| 5′ - CTG TAT TCA TAG CCG AAA CGA - 3′ | (VIIIb) |
| 5′ - CGC TGT GGT TGG TAT ATT TGT - 3′ | (IXa) |
| 5′ - GCC AAT TGC ACA AGT ACA AA - 3′ | (IXb) |
| 5′ - CAG CGG TTG GTA TAT TCG TT - 3′ | (Xa) |
| 5′ - AAA AAG AAG TGC ACA AAT AGA TGA - 3′ | (Xb) |
| 5′ - GTT TGA ATG GAG TCC GAC CG - 3′ | (XIa) |
| 5′ - CGG CGT ACT TGC TTC GGA GC - 3′ | (XIb) |
| 5′ - TGG GAT TAA CGG GCA GAG AC - 3′ | (XIIa) |
| 5′ - TTT CGC ATT CGG AGG CTT GG - 3′ | (XIIb) |
| 5′ - CGG TCG GAC TCC ATT CAA AC - 3′ | (XIIIa) |
| 5′ - GCT CCG AAG CAA GTA CGC CG - 3′ | (XIIIb) |
| 5′ - GTC TCT GCC CGT TAA TCC CA - 3′ | (XIVa) |
| 5′ - CCA AGC CTC CGA ATG CGA AA - 3′ | (XIVb). |

Where the assay method of the invention is concerned with testing for M. acerina and/or F. carotae, rather than Pythium, infection, the sample may conveniently be a vegetable or soil sample and the primers used are conveniently selected from 18- to 24-mers able to hybridize to sequences of formulae XIa to XIVb. Where however the assay method of the invention is concerned with testing for Pythium, rather than M. acerina and/or F. carotae, infection, the sample will preferably be a soil sample and the primers used are conveniently selected from 18- to 24-mers able to hybridize to sequences of formulae Ia to Xb. Where the assay method of the invention is concerned with testing for Pythium and M. acerina and/or F. carotae infection, the sample is preferably a soil sample and the primers used are conveniently selected from 18- to 24-mers able to hybridize to sequences of formulae Ia to Xb and XIa to XIVb.

In the assay method of the invention, the primer pair preferably comprises an 18- to 24-mer having the ability to hybridize to one of the oligonucleotide sequences of formulae Ia, Ib, IIa, IIb, IIIa, IIIb, IVa, IVb, Va, Vb, XIa, XIb, XIIa and XIIb. Even more preferably the primer pair comprises a pair of 18- to 24-mers having the ability to hybridize to a pair of the oligonucleotide sequences of formulae Ia and Ib, IIa and IIb, IIIa and IIIb, IVa and IVb, Va and Vb, XIa and XIb or XIIa and XIIb. For determination of Pythium infection the primer pair preferably comprises an 18- to 24-mer having the ability to hybridize to one of the oligonucleotide sequences of formulae Ia, Ib, IIa, IIb, IIIa, IIIb, IVa, IVb, Va and Vb. For determination of Pythium infection the primer pair especially preferably comprises a pair of 18- to 24-mers having the ability to hybridize to a pair of the oligonucleotide sequences of formulae Ia and Ib, IIa and IIb, IIIa and IIIb, IVa and IVb or Va and Vb. For determination of M. acerina and/or F. carotae infection the primer pair especially preferably comprises a pair of 18- to 24-mers having the ability to hybridize to a pair of the oligonucleotide sequences of formulae XIa and XIb or XIIa and XIIb. Less preferably the second primer of the primer pair may be a general primer that binds to all or substantially all fungal DNA. Such general primers, typically also 18- to 24-mers, are known and will still allow the polymerase chain reaction to function efficiently. Indeed such general primers are known which hybridize to DNA of all fungi, all oomycetes and all plants.

Examples of such general primers include:

| | |
|---|---|
| 5′ -TCC GTA GGT GAA CCT GCG G - 3′ | (A) |
| 5′ - GCT GCG TTC TTC ATC GAT GC - 3′ | (B) |
| 5′ - GCA TCG ATG AAG AAC GCA GC - 3′ | (C) |
| 5′ - TCC TCC GCT TAT TGA TAT GC - 3′ | (D) |
| 5′ - GGA AGT AAA AGT CGT AAC AAG G - 3′ | (E) |

General primers (A) (SEQ ID NO:29) and (E) (SEQ ID NO:33) are especially useful for use with specific primers which hybridize to sequences of formulae VIa, VIIa, VIIIa, IXa, Xa, XIIIa, XIVa, XIb and XIIb, or less preferably Ib, IIb, IIIb, IVb and Vb. General primer (D) (SEQ ID NO:32) is especially useful for use with specific primers which hybridize to sequences of formula VIb, VIIb, VIIIb, IXb, Xb, XIIIb, XIVb, XIa and XIIa, or less preferably Ia, IIa and IIIa.

General primer (C) (SEQ ID NO:31) is especially useful for use with specific primers which hybridize to sequences of formula Ia, IIa, IIIa, XIb and XIIb. General primer (B) (SEQ ID NO:30) is especially useful for use with specific primers which hybridize to sequences of formula Ib, IIb, IIIb, IVb, Vb, XIa and XIIa.

By “having the ability to hybridize to” is meant having the ability to anneal to DNA incorporating such a sequence at the site of that sequence under conditions under which primer annealing in the performance of a PCR reaction may be effected. Generally, PCR is effected under high stringency primer binding conditions, as detailed later below.

One primer is preferably a compound consisting of or comprising a sequence of formulae Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIa, XIIb, XIIIa, XIIIb, XIVa or XIVb or a derivative thereof in which up to 5 nucleotide residues: are omitted (deleted) or replaced by different residues; or are inserted; or are omitted (deleted) from or added to the 3′ or 5′ termini. In the case of such derivatives, preferably no more than one residue is omitted at a 3′ terminus and no more than 3 at a 5′ terminus, preferably no C residue is replaced by an A residue, preferably no more than 3 C or G residues are replaced, preferably no more than one omission or insertion within the listed sequence occurs and preferably any extension at the 3′ termini is 5′-CAACA-3, 5′-CCACC-3, 5′-TGCTG-3, 5′-ACAGG-3′, 5′-CCGGC-3, 5′-TTTGC-3, 5′-AGACA-3, 5′-AGAAG-3′, 5-CGAGA-3, 5′-GTTTG-3, 5′-GGCGC-3, 5′-GCCGA-3′, 5′-GGCTG-3, 5′-AGGCC-3, 5′-GGTCG-3, 5′-CCAAA-3′, 5′TTATG-3, 5′-AACAC-3, 5′-TATGC-3, 5′-CAGAT-3, 5′-GCGGG-3, 5′-GCAGC-3, 5′-GTGCA-3, 5′-ATTGT-3′, 5′-CCTTT-3, 5′-GCTGC-3, 5′-ACCCA-3′ or 5′-CAAAT-3′ or a fragment from the 5′ end thereof for Ia, Ib, Ia, IIb, IIIa, IIIb, IVa, IVb, Va, Vb, VIa, VIb, VIIa, VIIb, VIIIa, VIIIb, IXa, IXb, Xa, Xb, XIa, XIb, XIIa, XIIb, XIIIa, XIIIb, XIVa and XIVb respectively. More preferably, in such derivatives, no more than 3 residues are replaced or omitted, and particularly no more than 2 C or G residues are replaced.

In alternative preferred derivatives, none of the C or G residues are replaced or omitted (deleted). In further preferred derivatives any replacement, omission (deletion) or addition of nucleotides is made in the 5′ portion of the primer sequence, e.g. in the 5′ half of the primer sequence. Preferably 8 or more nucleotide residues, e.g. 8, 9 or 10 residues, at the 3′ end of the primers are not altered.

Fragments of such derivatives which have the ability to hybridise to sequences of formula Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIa, XIIb, XIIIa, XIIIb, XIVa or XIVb are also included.

“Substantially homologous” as used herein in connection with a nucleic acid sequence includes those sequences having a sequence homology or identity of approximately 60% or more, e.g. 70%, 75%, 80%, 85%, 90%, 95%, 98% or more, with a particular sequence and also functionally equivalent variants and related sequences modified by single or multiple base substitution, addition and/or deletion. By “functionally equivalent” in this sense is meant nucleotide sequences which have the ability to hybridise to sequences of formula Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIa, XIIb, XIIIa, XIIIb, XIVa or XIVb, in accordance with the definition above. Such functionally equivalent variants may include synthetic or modified nucleotide residues providing the hybridisation function of the primer is retained.

Sequences which “hybridise” as used herein in connection with the definition of derivative primers are those sequences which bind or anneal (hybridise) to a particular (specific) DNA sequence under conditions of low or preferably high stringency. Such conditions are well known and documented in the art. For example such sequences may hybridise to a particular DNA sequence under non-stringent conditions (e.g. 6×SSC, 50% formamide at room temperature) and can be washed under conditions of low stringency (e.g. 2×SSC, room temperature, more preferably 2×SSC, 42 C) or conditions of higher stringency (e.g. 2×SSC, 65 C) (where SSC=0.15M NaCl, 0.015M sodium citrate, pH 7.2).

Generally speaking, sequences which hybridise under conditions of high stringency are included within the scope of the invention.

For the detection of Pythium infection, one primer of the primer pair is preferably a compound consisting of or comprising a sequence of one of formulae Ia to Xb or a said derivative thereof. For the detection of M. acerina and/or F. carotae infection one primer is preferably a compound consisting of or comprising a sequence of one of formulae XIa to XIVb or a derivative thereof. For the detection of Pythium infection, preferably one of the primers is a compound consisting of or comprising a sequence of formula VIa, VIb, VIIa, VIIb, VIIIa, VIIIb, IXa, IXb, Xa or Xb or such a derivative thereof. More preferably the primer pair comprises the two compounds consisting of or comprising a sequence of formula VIa and VIb, VIIa and VIIb, VIIIa and VIIIb, IXa and IXb or Xa and Xb or such derivatives thereof.

For the detection of M. acerina and/or F. carotae infection, preferably one of the primers is a compound consisting of or comprising a sequence of formula XIIIa, XIIIb, XIVa, or XIVb or a such derivative thereof. More preferably the primer pair comprises two compounds consisting of or comprising sequences of formula XIIIa and XIIIb or XIVa and XIVb, or such derivatives thereof.

Especially preferably the primers comprise two or three pairs of compounds consisting of or comprising sequences of formulaes na and nb where n is VI to X, XIII, and XIV, e.g. VIa and VIb and XIIIa and XIIIb or VIa and VIb and XIVa and XIVb or XIIIa and XIIIb and XIVa and XIVb. In this way infection by two or three of Pythium, M. acerina and F. carotae may be detected.

The 18 to 24-mer primers may be prepared by conventional chemical techniques, e.g. solid state synthesis. As used herein a “primer pair” relates to two distinct primers with different sequences that may be utilized in any form of DNA amplification (including PCR) to amplify a fragment of DNA. The primers thus each anneal (bind or hybridize) to opposing (complementary) strands of the DNA to be amplified. The primer binding site flank the region to be amplified, thus ensuring that only the region of interest is amplified.

As used herein the term “primer” relates to an oligonucleotide which binds or anneals to a target DNA (or nucleic acid) sequence. Such a primer is a short polymer of nucleotides, as is generally, as defined above, 18 to 24 nucleotides in length, when used to prime DNA amplification. The term priming will be understood to include any annealing event which occurs between the oligonucleotide and the target nucleic acid sequence that provides a free 3′ OH end bound to the target in order to initiate synthesis of DNA amplification.

The “primer” as used herein may further be utilized as an oligonucleotide probe since it has the ability to specifically hybridize (or anneal) to the sequence to which it is complementary or substantially complementary as hereinbefore defined. It will be understood by those skilled in the art that such oligonucleotides are usually 13 to 35 nucleotides in length, i.e. 15 to 25, 20, 25, 30 or 35 nucleotides in length, but may also be shorter i.e. 8 to 15 nucleotides in length, i.e. 8, 9, 10, 11, 12, 13, 14 or 15 nucleotides in length.

It is especially preferred that two primer pairs be used in the method of the invention, one pair comprising primers hybridizing to sequences of formulae Ia and/or Ib (or less preferably VIa and/or VIb) and another comprising primers hybridizing to sequences of formulae IIa and/or IIb (or less preferably VIIa and/or VIIb), more preferably a further pair comprising primers hybridizing to sequences of formulae IIIa and/or IIIb (or less preferably VIIIa and/or VIIIb) is also used, still more preferably a still further pair comprising primers hybridizing to sequences of formulae IVa and/or IVb (or less preferably IXa and/or IXb) is used, most preferably five pairs of primers hybridizing to sequences of formulae Ia to Vb are used. The primer pairs of formulae Ia to Vb (or VIa to Xb) detect respectively infection by P. sulcatum, P. viola L, P. intermedium, P. sylvatium and P. violae/P. pareocandrum.

It is also especially preferred that two primer pairs be used in the method of the invention, one pair comprising primers hybridizing to sequences of formulae XIa and/or XIb (or less preferably XIIIa and/or XIIIb) and another comprising primers hybridizing to sequences of formulae XIIa and/or XIIb (or less preferably XIVa and/or XIVb), i.e. respectively to detect M. acerina and F. carotae infection. These two pairs may be used in addition to or in place of the two pairs mentioned in the previous paragraph.

Such use of two or more primer pairs may be simultaneous or, more preferably in separate PCR reactions on aliquots of the sample.

The primers are themselves novel compounds and form a further aspect of the invention.

Viewed from this aspect the invention provides an 18- to 24-mer oligonucleotide primer hybridizable to an oligonucleotide sequence selected from those of formulae Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIa, XIIb, XIIIa, XIIIb, XIVa and XIVb (e.g. one of formula Ia to Xb or XIa to XIVb).

Viewed from a still further aspect the invention provides a primer composition comprising a pair of 18- to 24-mer oligonucleotide primers at least one of which is hybridizable to an oligonucleotide sequence of formula Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIa, XIIb, XIIIa, XIIIb, XIVa and XIVb (e.g. one of formula Ia to Xb or XIa to XIVb) optionally together with a carrier.

In one embodiment, the composition of the invention preferably comprises a pair of 18- to 24-mer oligonucleotide primers hybridizable to the oligonucleotide sequences of formula Ia and Ib, IIa and IIb, IIIa and IIIb, IVa and IVb or Va and Vb, optionally two, three, four or five such pairs. In another embodiment, the composition of the invention preferably comprises a pair of 18- to 24-mer oligonucleotide primers hybridizable to the oligonucleotide sequences of formulae XIa and XIb and/or a pair of 18- to 24-mer oligonucleotide primers hybridizable to the oligonucleotide sequences of formulae XIIa and XIIb. In an especially preferred embodiment, the composition comprises a pair of 18- to 24-mer oligonucleotide primers hybridizable to the oligonucleotide sequences of formula Ia and Ib, IIa and IIb, IIIa and IIIb, IVa and IVb or Va and Vb, optionally two, three, four or five such pairs, as well as a pair of 18- to 24-mer oligonucleotide primers hybridizable to the oligonucleotide sequences of formulae XIa and XIb and/or a pair of 18- to 24-mer oligonucleotide primers hybridizable to the oligonucleotide sequences of formulae XIIa and XIIb.

For the detection phase of the method of the invention, it is possible to use labelled primers, e.g. radiolabelled or labelled with a chromophore or fluorophore or an enzyme. Such labelled versions of the primers of the invention and compositions containing them form further aspects of the invention.

Viewed from a yet still further aspect the invention provides a kit for the performance of the assay method of the invention, said kit comprising at least one primer pair according to the invention together with instructions for the performance of the assay method. Advantageously the kit also comprises a DNA-polymerase, e.g. Taq-polymerase, and especially advantageously the kit includes a set of components (e.g. chemical compositions) for DNA extraction.

The soil sample, approximately 0.5 g for each PCR reaction, is preferably taken from a larger sample, for example at least 100 g, more preferably at least 200 g, e.g. up to 1000 g, which has been mixed (e.g. by physical intermingling of the larger sample or by addition together of aliquots of different parts of the larger sample) so that the sample analysed is representative of the larger sample—this is in distinct contrast to conventional PCR-based DNA analysis of soil where such representative sampling is not effected. The sample may be taken from a single location or it may be the combination of samples from multiple locations in a growing area (e.g. a field). The separate analysis of multiple samples from different locations in a field is preferable but, for reasons of economy, analysis of a composite sample may be preferred.

The soil is preferably taken at a depth of up to 30 cm, especially 1 to 20 cm. Samples are also preferably taken from both the margins and the central section of the growing area, preferably at a distance of at least 3 m from the edge of the growing area (e.g. from a hedge, ditch, fence, track, etc).

Where the field is already in use in vegetable, e.g. carrot, production, the soil samples are advantageously taken from the soil within 10 cm, more preferably within 5 cm of the growing vegetables. Particularly conveniently, vegetables are uprooted and the soil on the uprooted vegetables is used for the assay.

We have found that humus in the soil reduced the accuracy of the assay method of the invention and thus pathogen DNA extraction from the soil samples preferably involves the following steps:
1) contact a sample of about 0.1 to 1 g, preferably about 0.5 g, soil taken from a mixed sample of at least 100 g, preferably at least 200 g, soil with a fungal cell lysing agent;
2) centrifuge at least 10000×g for at least 10 minutes and collect the supernatant;
3) contact the supernatant with a particulate DNA-binding agent;
4) centrifuge and collect the DNA-bearing particulate;
5) suspend the particulate in an aqueous solution of a chaotropic agent (e.g. an aqueous guanidine thiocyanate solution), centrifuge and collect the DNA-bearing particulate;
6) repeat step (5) at least once;
7) suspend the particulate in aqueous salt/ethanol wash solution, centrifuge and collect the DNA-bearing particulate;
8) repeat step (7) at least once;
9) suspend the particulate in an aqueous solution of a DNA-release agent;
10) centrifuge and collect the DNA-containing supernatant; and optionally
11) resuspend the particulate in an aqueous solution of a DNA-release agent, centrifuge and collect and combine the supernatant.

As compared with DNA-from-soil extraction using the commercially available kit FastDNA SPIN Kit for Soil (available from Qbiogene Inc/Bio 101 of Carlsbad, Calif., USA), this DNA extraction procedure involves a significantly longer post-lysis centrifugation, and repeated rinsing of the DNA-bearing particulate. In general also significantly larger volumes of release agent to free the DNA from the binding matrix should be used. Nonetheless the resultant procedure is one which provides good results for the full range of soil types in which vegetables are grown. The prior art extraction techniques in comparison are very sensitive to the soil type under investigation.

Thus viewed from a further aspect the invention provides a process for the extraction of nucleic acid (e.g. DNA) from soil which process comprises:
1) contact a sample of about 0.1 to 1 g, preferably about 0.5 g, soil taken from a mixed sample of at least 100 g, preferably at least 200 g, soil with a fungal cell lysing agent (e.g. a ceramic and silica particulate);
2) centrifuge at least 10000×g for at least 10 minutes and collect the supernatant;
3) contact the supernatant with a particulate DNA-binding agent;
4) centrifuge and collect the DNA-bearing particulate;
5) suspend the particulate in an aqueous solution of a chaotropic agent (e.g. an aqueous guanidine thiocyanate solution), centrifuge and collect the DNA-bearing particulate;
6) repeat step (5) at least once;
7) suspend the particulate in aqueous salt/ethanol wash solution (generally with a water/ethanol volume ratio of about 1:10), centrifuge and collect the DNA-bearing particulate;
8) repeat step (7) at least once;
9) suspend the particulate in an aqueous solution of a DNA-release agent;
10) centrifuge and collect the DNA-containing supernatant; and optionally
11) resuspend the particulate in an aqueous solution of a DNA-release agent (e.g. DNase in pyrogen-free water), centrifuge and collect and combine the supernatant.

Viewed from a further aspect the invention provides a kit for nucleic acid (e.g. DNA) extraction from soil, which kit comprises:
i) a fungal cell lysing agent;
ii) a DNA-binding particulate;
iii) an aqueous solution of a chaotropic agent (e.g. guanidine thiocyanate);
iv) an aqueous solution of salt and ethanol; and
v) an aqueous solution of a DNA-release agent;

together with instructions for the use of said kit in the process of the invention.

Where the sample under analysis is of vegetable tissue rather than soil, it is preferably surface tissue, in particular root (or tuber) surface tissue. Such a sample may be taken for example by peeling the root (or tuber surface) optionally after washing, wiping or rinsing to remove soil. Such samples may be taken at any stage during growth or storage but will preferably (for analysing for Pythium ssp. involved in cavity spot) be taken from 2 weeks after sowing up to harvesting, more preferably 4 weeks after sowing up to harvesting. Where, as is preferred, the vegetable is carrot, we have found that unsaturated organic compounds in the carrot root reduced the accuracy of the assay method of the invention and thus pathogen DNA extraction from vegetable tissue samples preferably involves the following steps:
i) contact at least 20 mg of dry powdered plant tissue (preferably surface tissue such as peel) with at least 5 μL/mg dry tissue of an aqueous fungal cell lysing agent;
ii) incubate;
iii) mix with at least 4.5 μL/mg dry tissue of an aqueous solution of a protein and polysaccharide precipitating agent;
iv) centrifuge and collect DNA-containing supernatant;
v) filter;
vi) contact DNA-containing filtrate with a DNA-binding substrate and centrifuge;
vii) wash the DNA-carrying substrate with an aqueous ethanolic solution, centrifuge and remove the liquid phase;
viii) repeat step (vii) at least once;
ix) dry the DNA-carrying substrate; and
x) contact the substrate with an aqueous solution of a DNA release agent, centrifuge and collect the DNA-containing supernatant.

As compared with DNA-from-plant-tissue extraction using the commercially available GenElute Plant Genomic DNA kit (available from Sigma), this DNA extraction procedure involves the use of dry powdered plant tissue, larger volumes of lysing and precipitation solutions and drying of the DNA-carrying substrate to remove ethanol. Nonetheless the procedure does provide significantly better results and thus viewed from a further aspect the invention provides a process for the extraction of pathogen DNA from host vegetable tissue, which process comprises:
i) contact at least 20 mg of dry powdered plant tissue (preferably surface tissue such as peel) with at least 5 μL/mg dry tissue of an aqueous fungal cell lysing agent;
ii) incubate;
iii) mix with at least 4.5 μL/mg dry tissue of an aqueous solution of a protein and polysaccharide precipitating agent;
iv) centrifuge and collect DNA-containing supernatant;
v) filter;
vi) contact DNA-containing filtrate with a DNA-binding substrate and centrifuge;
vii) wash the DNA-carrying substrate with an aqueous ethanolic solution, centrifuge and remove the liquid phase;
viii) repeat step (vii) at least once;
ix) dry the DNA-carrying substrate; and
x) contact the substrate with an aqueous solution of a DNA release agent, centrifuge and collect the DNA-containing supernatant.

Viewed from a still further aspect the invention provides a kit for pathogen DNA extraction from host vegetable tissue, which kit comprises:
a) a fungal cell lysing agent;
b) an aqueous solution of a protein and polysaccharide precipitating solution;
c) a DNA-binding substrate;
d) an aqueous ethanolic wash solution; and
e) an aqueous solution of a DNA release agent;
   together with instructions for the use of said kit for pathogen DNA extraction from host vegetable tissue.

In these techniques, the fungal cell lysing agent may for example be an enzyme (e.g. L1393 or L1412 from Sigma) or a buffered surfactant (e.g. cetyltrimethylammonium bromide, N-lauroylsarcosine or sodium dodecyl sulphate). Alternatively, mechanical means such as grinding in liquid nitrogen, may be used.

Proteins, polysaccharides and nucleic acids can be separated in these techniques by different strategies. Thus proteins can be precipitated leaving the nucleic acid in solution, for example by adjusting the osmolality of the solution, e.g. by the addition of salts, generally high concentration salt solutions, for example 3M sodium acetate. Proteins can alternatively be extracted using organic solvents such as chloroform or phenol.

DNA extracted from the samples will typically be purified before being subjected to PCR using the primers of the invention. This can be effected in conventional fashion, e.g. chromatographically. Thus for example Micro Bio-Spin chromatography columns (available from BioRad, Hercules, Calif., USA) may be used together with insoluble polyvinylpolypyrrolidone powder (e.g. P6755 from Sigma) to purify the DNA.

For the primer pair which hybridizes to the sequences of formula Ia and Ib, the amplified section of DNA is about 646 bp, for the pair which hybridizes to the sequences of formulae IIa and IIb, the amplified section of DNA is about 352 bp, for the pair which hybridizes to the sequences of formulae IIIa and IIIb, the amplified section of DNA is about 380 bp, for the pair which hybridizes to the sequences of formulae IVa and IVb, the amplified section of DNA is about 330 bp, and for the pair which hybridizes to the sequences of formulae Va and Vb, the amplified section of DNA is about 329 bp. For the primer pair which hybridizes to the sequences of formula XIa and XIb, the amplified section of DNA is about 294 bp while for the pair which hybridizes to the sequences of formulae XIIa and XIIb, the amplified section of DNA is about 359 bp.

The PCR reaction itself can again be effected conventionally, e.g. using the primer pair, the four deoxynucleotide triphosphates (hereinafter “nucleotides”) and a heat stable DNA polymerase (e.g. Taq polymerase, available from Roche). Generally at least 25, more preferably 30 to 50, cycles of the PCR reaction will be sufficient.

It will be understood that any suitable nucleotides may be used, including chemically modified nucleotides and analogues or derivatives thereof, including labelled nucleotides.

The amplified DNA, if present, may then be detected by conventional techniques, e.g. gel separation or hybridization to labelled probes (for example radiolabelled or chromophore/fluorophore labelled probes). Where labelled probes are used, these may typically comprise labelled versions of one of the primer pair or labelled oligonucleotides able to hybridize specifically to the PCR-amplified fragment. In this embodiment, the PCR product is typically detected by a photodetector during PCR amplification or taken up by a porous substrate which is then treated with the labelled probe and rinsed, whereafter the signal from the probe retained on the substrate may be detected, e.g. photometrically or using a radiation detector. Where more than one primer pair is used in the PCR reaction, more than one probe will likewise be used and these may be labelled in the same or different fashion, e.g. using labels with different characteristic absorption or emission energies or wavelengths.

Alternatively, PCR may be effected using one or more labelled nucleotides, such as those labelled with a fluorophore, and the presence of such a label in the amplification reaction mixture detected by standard means.

The detection of the amplified DNA may be used to provide a qualitative, semi-quantitative or quantitative indication of the pathogen infestation of the soil sample, e.g. a value in cells per unit weight or an indication that the pathogen content of the soil is above or below a predetermined threshold value, e.g. boundary value for the decision to plant or not plant a particular vegetable crop or the decision to apply or not apply a fungicide.

In a particularly preferred embodiment of the method of the invention, aliquots of the soil sample are also tested in similar fashion for the presence of the fungal pathogens responsible for other vegetable root disease, e.g. ring rot (caused by Phytopthora species, in particular P. megasperma), grey mould (caused by Botrytis cinerea), Sclerotina rot (caused by Sclerotinia sclerotiorum), Chalaropsis rot (caused by Chalaropsis thielaviodes), and other diseases caused by Alternaria dauci, Cercospora carotae and Rhizoctonia solani.

If the method of the invention shows a carrot crop to be infected with M. acerina and/or F. carotae, the harvested crop should be consumed or processed (e.g. cooked, canned or bottled) within about 4 weeks.

The primer pairs used in the assay method of the invention clearly should not hybridize to the DNA of the vegetable (e.g. carrot) itself. While the method of the invention is particularly suited for use on soil from fields in which carrots are to be grown or are growing, it is also more generally applicable to fields for vegetable (in particular root vegetable) and potato production, especially parsnip, celery, lettuce, brassica and potato.

In place of the primers Ia to Xb it is possible to use in the method of the invention further primers which hybridize specifically to DNA of a Pythium species selected from Pythium violae/P. pareocandrum like, P. intermedium, P. sylvatium, P. sulcatum, P. sulcatum like and P. viola L. By specific hybridization it is meant that the primers are capable of being used to amplify DNA from the particular Pythium species in a PCR reaction but not capable of being used to amplify DNA from a non-pathogenic Pythium species or carrot DNA. Typically, specificity may be tested for by checking against carrot DNA and DNA from the deposited Pythium strains such as P. angustatum CBS 676.95 and P. monospermum CBS 790.95. These strains are deposited at Centraalbureau voor Schimmelcultures and are publicly available (http://www.cbs.knaw.nl/address/index.htm). The use of such primers in the method and kits of the invention in place of primers of formulae Ia to Xb is considered to fall within the scope of the present invention.

While PCR amplification of fungal DNA using the primers described herein will (in the case of an infected sample) yield oligonucleotides which can be detected on a gel, other routine methods for oligonucleotide detection may be used. Thus for example in the PCR reaction labelled nucleotides may be used resulting in the oligonucleotide product being itself labelled. When the oligonucleotide is separated from the unreacted nucleotides (e.g. chromatographically), it may be detected by detection of the label (e.g. a radiolabel or a chromophore or fluorophore). A further method of DNA fragment detection is to use a substrate (or solid support) on which a “primer” capable of capturing the fragments is immobilized. The bound fragments may then be detected, e.g. by surface plasmon resonance or by detection of a label where PCR has been effected using labelled nucleotides. In such an embodiment, it will be understood that the primers are acting as capturing oligonucleotide probes by hybridizing (or annealing) to their complementary sequence.

In one embodiment, where the primers of the invention are utilized to capture complementary DNA fragments, the bound DNA may be detected using any known method of the art. Such methods include a competition assay using labelled or unlabelled fragments which compete for binding to the primers with the DNA fragments. Surface Plasmin Resonance may be used to detect the unlabelled fragments binding to the primers. Alternatively, sandwich assays are envisaged using labelled probes which bind to the DNA fragment and the presence of unbound or bound probes are detected, thus indicating whether the DNA fragment is present.

In an alternative aspect, the primers may be used in the detection step to capture the oligonucleotide product of the PCR reaction. In this aspect, the primers are immobilized on a substrate, e.g. a solid support such as a plate, rod, bead, etc. and this causes the oligonucleotide to be immobilized too, since it is complementary to the primer sequence. The immobilized oligonucleotide can then be detected by standard means, e.g. by detection of a label where labelled nucleotides have been used in the PCR reaction or by surface plasmon resonance. In this aspect, the PCR reaction can be carried out using the primers described herein but alternatively general (i.e. universal) primers may instead be used as the immobilized specific primers will serve to separate out from the PCR product those oligonucleotides indicative of infection. The primers may be bound to the substrate surface by conventional means, e.g. as described by Laassri et al in J. Virological Methods 112: 67-78 (2003) or Keramas et al in Molecular and Cellular Probes 17: 187-196 (2003). Desirably, the substrate will be provided with capture primers at a plurality of locations, e.g. with different primers at different (known) sites and preferably with at least one site carrying a general (universal) primer to act as a control. Most preferably, the substrate will take the form of a microarray plate, e.g. with different primers printed onto the different sites of the array.

Thus viewed from a further aspect the invention provides an array method for detecting fungal infection of soil or vegetables by pathogenic fungal species, in particular M. acerina, F. carotae and Pythium species, said method comprising:

obtaining a sample of soil or vegetable; treating said sample to lyse fungal cells therein; using an oligonucleotide primer pair, effecting a polymerase chain reaction on DNA released by lysis of the fungal cells; contacting the DNA fragments generated by said polymerase chain reaction with a substrate having immobilized thereon a primer which comprises an 18- to 24-mer having the ability to hybridize to one of the oligonucleotide sequences of formulae Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIa, XIIb, XIIIa, XIIIb, XIVa and XIVb:

| | |
|---|---|
| 5′ - TCA CTT GTG GGG TAA AGA AGA - 3′ | (Ia) |
| 5′ - AGA CCA CAA TAA AGC GGC - 3′ | (Ib) |
| 5′ - AGT CCC GCA CAC ACA CAT - 3′ | (IIa) |
| 5′ - ACT TCT CTC TTT GGG GAG TGG - 3′ | (IIb) |
| 5′ - TTC GTT CAG CCT CTG CAT - 3′ | (IIIa) |
| 5′ - TCG TTT CGG CTA TGA ATA CAG - 3′ | (IIIb) |
| 5′ - ACA AAT ATA CCA ACC ACA GCG - 3′ | (IVa) |
| 5′ - TTT GTA CTT GTG CAA TTG GC - 3′ | (IVb) |
| 5′ - AAC GAA TAT ACC AAC CGC TG - 3′ | (Va) |
| 5′ - TCA TCT ATT TGT GCA CTT CTT TTT - 3′ | (Vb) |
| 5′ - TCT TCT TTA CCC CAC AAG TGA - 3′ | (VIa) |
| 5′ - GCC GCT TTA TTG TGG TCT - 3′ | (VIb) |
| 5′ - ATG TGT GTG TGC GGG ACT - 3′ | (VIIa) |
| 5′ - CCA CTC CCC AAA GAG AGA AGT - 3′ | (VIIb) |
| 5′ - ATG CAG AGG CTG AAC GAA - 3′ | (VIIIa) |
| 5′ - CTG TAT TCA TAG CCG AAA CGA - 3′ | (VIIIb) |
| 5′ - CGC TGT GGT TGG TAT ATT TGT - 3′ | (IXa) |
| 5′ - GCC AAT TGC ACA AGT ACA AA-3′ | (IXb) |
| 5′ - CAG CGG TTG GTA TAT TCG TT - 3′ | (Xa) |
| 5′ - AAA AAG AAG TGC ACA AAT AGA TGA - 3′ | (Xb) |
| 5′ - GTT TGA ATG GAG TCC GAC CG - 3′ | (XIa) |
| 5′ - CGG CGT ACT TGC TTC GGA GC - 3′ | (XIb) |
| 5′ - TGG GAT TAA CGG GCA GAG AC - 3′ | (XIIa) |
| 5′ - TTT CGC ATT CGG AGG CTT GG - 3′ | (XIIb) |
| 5′ - CGG TCG GAC TCC ATT CAA AC - 3′ | (XIIIa) |
| 5′ - GCT CCG AAG CAA GTA CGC CG - 3′ | (XIIIb) |
| 5′ - GTC TCT GCC CGT TAA TCC CA - 3′ | (XIVa) |
| 5′ - CCA AGC CTC CGA ATG CGA AA - 3′ | (XIVb); |

and detecting DNA fragments binding to said primer.

Viewed from a further aspect the invention comprises a substrate, e.g. a microarray plate, having immobilized thereon at least one 18- to 24-mer oligonucleotide primer hybridizable to an oligonucleotide sequence selected from those of formulae Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIA, XIIb, XIIIa, XIIIb, XIVa and XIVb. The invention also provides a kit for the performance of the assay method comprising a substrate according to the invention and instructions for the performance of the assay method.

The invention will now be illustrated further by the following non-limiting Examples.

### EXAMPLES 1 TO 10

### Primers of Formula VIa to Xb

These were ordered by formula and prepared commercially by Eurogentec, Serang, Belgium using conventional methods. Alternatively these may be prepared on a support matrix using a Pharmacia Gene Assembler Plus instrument. The primers produced are then deprotected and cleared from the support matrix by overnight incubation at 55° C. in 1 mL ammonia. Blocking groups and ammonia may be removed by chromatography on a Pharmacia NAP 10 column with the primer being eluted in 1 mL water. Primer concentration can then be estimated spectrophotometrically using the factor 1 AU=20 μg mL⁻¹ at 260 nm.

### EXAMPLE 11

### DNA Extraction from Soil

A FastDNA SPIN kit for Soil (available from Qbiogene Inc/Bio 101) is used in this Example. A soil sample is collected and treated as follows:
1. Add 300-500 mg of soil to Multimix Tissue matrix Tube and place on ice. Process in FastPrep instrument for 20 seconds at speed 4.5 and place on ice. Add 980 μl Sodium Phosphate Buffer and 122 μl MT Buffer and process in FastPrep instrument for 30 seconds at speed 5.5 and place on ice
2. Centrifuge at 14,000×g for 15 minutes and place on ice
3. Transfer supernatant to new tubes (1.5 ml tubes) and add 250 μl PPS
4. Mix by inverting the tubes by hand 10 times and centrifuge at 14,000×g for 5 minutes
5. Transfer supernatant to new tubes (2 ml tubes), add 1 ml RESUSPENDED Binding Matrix Suspension and invert by hand for 2 minutes
6. Centrifuge at 14,000×g for 5 seconds and discharge supernatant
7. Resuspend in 1 ml of 5.5M Guanidine Thiocyanate
8. Centrifuge at 14,000×g for 5 seconds and discharge supernatant
9. Resuspend in 600 μl of 5.5M Guanidine Thiocyanate and transfer to new tubes with Spin Filters
10. Centrifuge at 14,000×g for 1 minute and empty catch tube
11. Add 500 μl SEWS-M (aqueous salt/ethanol solution) to the Spin Filter (Wash 1) and resuspend matrix
12. Centrifuge at 14,000×g for 1 minute and empty catch tube
13. Add 500 μl SEWS-M to the Spin Filter (Wash 2) and resuspend matrix
14. Centrifuge at 14,000×g for 1 minute and empty catch tube
15. Centrifuge 14,000×g for 2 minutes
16. Place Spin Filters in new catch tubes and air dry for 5 minutes
17. Add 100 μl DES and resuspend matrix
18. Centrifuge at 14,000×g for 1 minutes
19. Store in fridge or at −20° C.

### EXAMPLE 12

### DNA Extraction from Carrot Peel

A GenElute Plant Genomic DNA kit (available from Sigma) is used in this Example. The carrot tissue sample is prepared by rinsing the carrot in water then peeling one third of the length of the top and tip. The peel is freeze dried then ground to powder. DNA extraction then proceeds as follows:
1. Place about 50 mg dried carrot tissue powder in a microfuge tube
2. Add 700 μl of Lysis Solution Part A and 100 μl of Lysis Solution Part B
3. Mix by vortexing and inversion and incubate at 65° C. for 10 minutes with occasional inversions
4. Add 260 μl Precipitation Solution and mix by inversions
5. Place on ice for 5 minutes
6. Centrifuge at 14,000×g for 5 minutes (to pelletize cellular debris, proteins and polysaccharides)
7. Carefully transfer supernatant to a filtration column (BLUE filter in a collection tube)
8. Centrifuge at 14,000×g for 1 minute and discard the filtration column
9. Add 700 μl of Binding Solution and mix by pipetting up and down 3 times
10. Transfer about 700 μl to a Nucleic Acid binding column (COLORLESS insert with a RED O-RING in a collection tube)
11. Centrifuge at 14,000×g for 1 minute and empty the collection tube
12. Transfer the remainder of the liquid from step (9) to the Nucleic Acid binding column
13. Centrifuge at 14,000×g for 1 minute and discard the collection tube
14. Place column in a new collection tube and add 500 μl diluted Washing Solution (Wash 1)
15. Centrifuge at 14,000×g for 1 minute and empty collection tube
16. Add 500 μl diluted Washing Solution (Wash 2)
17. Centrifuge at 14,000×g for 1 minute
18. Transfer column to new collection tube and air dry for 5 minutes
19. Elute DNA with 100 μl pre-warmed (65° C.) Elution solution by centrifugation at 14,000×g for 1 minute.

### EXAMPLE 13

### DNA Purification

For this Example, Micro Bio-Spin Chromatography columns (available from BioRad) and insoluble polyvinylpolypyrrolidone powder (P6755 from Sigma) are used. DNA purification is then effected as follows:
1. Place column in a 1.5 ml centrifuge tube
2. Fill column with polyvinylpolypyrrolidone powder to 1 mm below the edge and add 400 ml double distilled H₂O
3. Centrifuge at 4,000 rpm (tabletop centrifuge) for 5 minutes
4. Transfer column to new 1.5 ml centrifuge tube and add DNA extract from Example 11 or 12
5. Centrifuge at 4,000 rpm (tabletop centrifuge) for 4 minutes and discharge column
6. Store DNA at −20° C.

### EXAMPLES 14 TO 18

### DNA Amplification Using the Primers of Examples 1 to 10

The reactions are done in a total volume of 25 μl and the PCR reaction mixture is prepared as follows for the primers of Examples 1 to 8:

| | |
|---|---|
| 15.87 μl | H₂O |
| 2.5 μl | 10× PCR buffer containing 15 mM MgCl₂ (Roche) |
| 2.0 μl | dNTP 2.5 mM |
| 2.5 μl | BSA (bovine serum albumin) 1 mg/ml |
| 0.5 μl | Forward primer (50 pmol/μl) |
| 0.5 μl | Reverse primer (50 pmol/μl) |
| 0.13 μl | Taq DNA polymerase (Roche) 5U/μl |
| 1.0 μl | DNA template |

For the primer pair of Examples 9 and 10, 14.37 μL H₂O is used, and 1.5 μL 25 mM MgCl₂ is additionally used.

The PCR program used for the primers of Examples 1 to 6 is:

| | |
|---|---|
| 1. Denaturation | 94° C. 5 min |
| 2. 30 cycles of | 94° C. 20 sec, 60° C. 30 sec, 72° C. 30 sec |
| 3. Terminal elongation | 72° C. 2 min |
| 4. Storage | 4° C. |

The PCR program used for the primers of Examples 7 to 10 is:

| | |
|---|---|
| 1. Denaturation | 94° C. 5 min |
| 2. 30 cycles of | 94° C. 30 sec, 56° C. 30 sec, 72° C. 30 sec |
| 3. Terminal elongation | 72° C. 2 min |
| 4. Storage | 4° C. |

After amplification, 10 μl of the PCR product are added to 2 μl DNA loading buffer and run on a 1.2% agarose gel in 1×TBE or 1×TAE buffer at 100V for 45 minutes.

In Examples 14 to 18, the forward and reverse primers are the primers of formulae VIa and VIb of Examples 1 and 2, VIIa and VIIb of Examples 3 and 4, VIIIa and VIIIb of Examples 5 and 6, IXa and IXb of Examples 7 and 8 and Xa and Xb of Examples 9 and 10 respectively.

### EXAMPLE 19

### Sensitivity

The primer pairs of Examples 1/2, 3/4, 5/6, 7/8, and 9/10 were tested against DNA extracted from Pythium intermedium, Pythium sulcatum, Pythium sulcatum like, Pythium angustatum, Pythium aphanidermatum, Pythium aquatile, Pythium coloratum, Pythium connatum, Pythium deliense, Pythium dissotocum, Pythium irregulare, Pythium mamilatum, Pythium middletonii, Pythium monospermum, Pythium myriotylum, Pythium rostratum, Pythium tracheiphilum, Pythium torulosum, Pythium ultimum, Pythium group F, Pythium group T, Pythium group HS, Pythium sylvatium, Pythium violae L, Pythium violae/Pythium pareocandrum like, Phytophthora infestans, Phytophthora cryptogea, Stemphyllium sp., Verticillium sp., Fusarium sp., Rhizoctonia sp., Rhizoctonia solani, Cylindrocarpon sp., Botrytis sp., healthy carrot, Mycocentrospora acerina and Fibularhizoctonia carotea.

The results are set out in Table 1 below.

TABLE 1

| Species | Example 1/2 | Example 3/4 | Example 5/6 | Example 7/8 | Example 9/10 |
|---|---|---|---|---|---|
| Pythium intermedium | − | − | + | − | − |
| Pythium sulcatum | + | − | − | − | − |
| Pythium sulcatum like | + | − | − | − | − |
| Pythium angustatum | − | − | − | − | − |
| Pythium aphanidermatum | − | − | − | − | − |
| Pythium aquatile | − | − | − | − | − |
| Pythium coloratum | − | − | − | − | − |
| Pythium connatum | − | − | − | − | − |
| Pythium deliense | − | − | − | − | − |
| Pythium dissotocum | − | − | − | − | − |
| Pythium irregulare | − | − | − | − | − |
| Pythium mamilatum | − | − | − | − | − |
| Pythium middletonii | − | − | − | − | − |
| Pythium monospermum | − | − | − | − | − |
| Pythium myriotylum | − | − | − | − | − |
| Pythium rostratum | − | − | − | − | − |
| Pythium tracheiphilum | − | − | − | − | − |
| Pythium torulosum | − | − | − | − | − |
| Pythium ultimum | − | − | − | − | − |
| Pythium group F | − | − | − | − | − |
| Pythium group T | − | − | − | − | − |
| Pythium group HS | − | − | − | − | − |
| Pythium sylvatium | − | − | − | + | − |
| Pythium violae L | − | + | − | − | − |
| Pythium violae/ | − | − | − | − | + |
| Pythium pareocandrum like | | | | | |
| Phytophthora infestans | − | − | − | − | − |
| Phytophthora cryptogea | − | − | − | − | − |
| Stemphyllium sp. | − | − | − | − | − |
| Verticillium sp. | − | − | − | − | − |
| Fusarium sp. | − | − | − | − | − |
| Rhizoctonia sp. | − | − | − | − | − |
| Rhizoctonia solani | − | − | − | − | − |
| Cylindrocarpon sp. | − | − | − | − | − |
| Botrytis sp | − | − | − | − | − |
| Healthy carrot | − | − | − | − | − |
| Mycocentrospora acerina | − | − | − | − | − |
| Fibularhizoctonia carotea | − | − | − | − | − |

− = no DNA amplification
+ = DNA amplification

### EXAMPLES 20 TO 23

### Primers of Formulae XIIIa to XIVb

These were ordered by formula and prepared commercially by Eurogentec, Serang, Belgium using conventional methods. Alternatively these may be prepared on a support matrix using a Pharmacia Gene Assembler Plus instrument. The primers produced are then deprotected and cleaned from the support matrix by overnight incubation at 55° C. in 1 mL ammonia. Blocking groups and ammonia may be removed by chromatography on a Pharmacia NAP 10 column with the primer being eluted in 1 mL water. Primer concentration can then be estimated spectrophotometrically using the factor 1 AU=20 μg mL⁻¹ at 260 nm.

### EXAMPLES 24 AND 25

### DNA Amplification

The reactions are done in a total volume of 25 μl and the PCR reaction mixture is prepared as follows:

| | |
|---|---|
| 13.75 μl | H₂O |
| 2.5 μl | 10× PCR buffer containing 15 mM MgCl₂ (Roche) |
| 2.5 μl | dNTP 2 mM |
| 2.5 μl | BSA (bovine serum albumin) 1 mg/ml |
| 1.25 μl | Forward primer (20 pmol/μl) |
| 1.25 μl | Reverse primer (20 pmol/μl) |
| 0.25 μl | Taq DNA polymerase (Roche) 5U/μl |
| 1.0 μl | DNA template |

The PCR program used is:

| | |
|---|---|
| 1. Denaturation | 94° C. 5 min |
| 2. 45 cycles of | 94° C. 20 sec, 62° C. 30 sec, 72° C. 30 sec |
| 3. Terminal elongation | 72° C. 2 min |
| 4. Storage | 4° C. |

After amplification, 10 μl of the PCR product are added to 2 μl DNA loading buffer and run on a 1.2% agarose gel in 1×TBE or 1×TAE buffer at 100V for 45 minutes.

In Example 24, the forward and reverse primers are the primers of formulae XIIIa and XIIIb of Examples 20 and 21. In Example 25, the forward and reverse primers are the primers of formulae XIVa and XIVb of Examples 22 and 23.

The reaction mixture may alternatively comprise 15.87 μl water, 2.5 μl buffer (as above), 2.0 μl dNTP 2.5 mM, 0.5 μl forward primer (50 pmol/l), 0.5 μl reverse primer (50 pmol/l), 0.13 μl Taq DNA polymerase (as above), 1.0 μl DNA template and 2.5 μl BSA (as above) and the product may be run on a 1% agarose gel as described above.

### EXAMPLE 26

### Sensitivity

The primer pairs of Examples 20/21 and 22/23 were tested against DNA extracted from Pythium sylvatium, Pythium violae L, Pythium violae/Pythium pareocandrum like, Pythium irregulare, Pythium ultimum, Phytophthora infestans, Phytophthora megasperma, Stemphyllium sp., Verticillium sp., Fusarium “powdery poae”, Fusarium sporotrichioides, Fusarium avenaceum, Fusarium sp., Microdoccium nivale, Rhizoctonia sp., Rhizoctonia solani, Cylindrocarpon sp., Botrytis sp, healthy carrot, M. acerina and F. carotea.

The results are set out in Table 2 below.

TABLE 2

| | Example | Example |
|---|---|---|
| Species | 20/21 | 22/23 |
| M. acerina | + | − |
| F. carotea | − | + |
| Healthy carrot | − | − |
| Pythium sylvatium | − | − |
| Pythium violae L | − | − |
| Pythium violae/Pythium | − | − |
| pareocandrum like | | |
| Pythium irregulare | − | − |
| Pythium ultimum | − | − |
| Phytophthora infestans | − | − |
| Phytophthora megasperma | − | − |
| Stemphyllium sp. | − | − |
| Verticillium sp. | − | − |
| Fusarium “powdery poae” | − | − |
| Fusarium sporotrichioides | − | − |
| Fusarium avenaceum | − | − |
| Fusarium sp. | − | − |
| Microdoccium nivale | − | − |
| Rhizoctonia sp. | − | − |
| Rhizoctonia solani | − | − |
| Cylindrocarpon sp. | − | − |
| Botrytis sp. | − | − |

− = no DNA amplification
+ = DNA amplification

1. An assay method for detecting fungal infection of soil or vegetables by pathogenic fungal species, in particular M. acerina, F. carotae and Pythium species, said method comprising:
obtaining a sample of soil or vegetable; treating said sample to lyse fungal cells therein; using an oligonucleotide primer pair, effecting a polymerase chain reaction on DNA released by lysis of the fungal cells; and detecting DNA fragments generated by said polymerase chain reaction;
wherein said primer pair comprises an 18- to 24-mer having the ability to hybridize to one of the oligonucleotide sequences of formulae Ia (SEQ ID NO:1), Ib (SEQ ID NO:2), IIa (SEQ ID NO:3), IIb (SEQ ID NO:4), IIIa (SEQ ID NO:5), IIIb (SEQ ID NO:6), IVa (SEQ ID NO:7), IVb (SEQ ID NO:8), Va (SEQ ID NO:9), Vb (SEQ ID NO:10), VIa (SEQ ID NO:11), VIb (SEQ ID NO:12), VIIa (SEQ ID NO:13), VIIb (SEQ ID NO:14), VIIIa (SEQ ID NO:15), VIIIb (SEQ ID NO:16), IXa (SEQ ID NO:17), IXb (SEQ ID NO:18), Xa (SEQ ID NO:19), Xb (SEQ ID NO:20), XIa (SEQ ID NO:21), XIb (SEQ ID NO:22), XIIa (SEQ ID NO:23), XIIb (SEQ ID NO:24), XIIIa (SEQ ID NO:25), XIIIb (SEQ ID NO:26), XIVa (SEQ ID NO:27) and XIVb (SEQ ID NO:28):

| | |
|---|---|
| 5′ - TCA CTT GTG GGG TAA AGA AGA - 3′ | (Ia) |
| 5′ - AGA CCA CAA TAA AGC GGC - 3′ | (Ib) |
| 5′ - AGT CCC GCA CAC ACA CAT - 3′ | (IIa) |
| 5′ - ACT TCT CTC TTT GGG GAG TGG - 3′ | (IIb) |
| 5′ - TTC GTT CAG CCT CTG CAT - 3′ | (IIIa) |
| 5′ - TCG TTT CGG CTA TGA ATA CAG - 3′ | (IIIb) |
| 5′ - ACA AAT ATA CCA ACC ACA GCG - 3′ | (IVa) |
| 5′ - TTT GTA CTT GTG CAA TTG GC - 3′ | (IVb) |
| 5′ - AAC GAA TAT ACC AAC CGC TG - 3′ | (Va) |
| 5′ - TCA TCT ATT TGT GCA CTT CTT TTT - 3′ | (Vb) |
| 5′ - TCT TCT TTA CCC CAC AAG TGA- 3′ | (VIa) |
| 5′ - GCC GCT TTA TTG TGG TCT- 3′ | (VIb) |
| 5′ - ATG TGT GTG TGC GGG ACT- 3′ | (VIIa) |
| 5′ - CCA CTC CCC AAA GAG AGA AGT- 3′ | (VIIb) |
| 5′ - ATG CAG AGG CTG AAC GAA- 3′ | (VIIIa) |
| 5′ - CTG TAT TCA TAG CCG AAA CGA- 3′ | (VIIIb) |
| 5′ - CGC TGT GGT TGG TAT ATT TGT- 3′ | (IXa) |
| 5′ - GCC AAT TGC ACA AGT ACA AA-3′ | (IXb) |
| 5′ - CAG CGG TTG GTA TAT TCG TT- 3′ | (Xa) |
| 5′ - AAA AAG AAG TGC ACA AAT AGA TGA - 3′ | (Xb) |
| 5′ - GTT TGA ATG GAG TCC GAC CG - 3′ | (XIa) |
| 5′ - CGG CGT ACT TGC TTC GGA GC - 3′ | (XIb) |
| 5′ - TGG GAT TAA CGG GCA GAG AC - 3′ | (XIIa) |
| 5′ - TTT CGC ATT CGG AGG CTT GG - 3′ | (XIIb) |
| 5′ - CGG TCG GAC TCC ATT CAA AC - 3′ | (XIIIa) |
| 5′ - GCT CCG AAG CAA GTA CGC CG - 3′ | (XIIIb) |
| 5′ - GTC TCT GCC CGT TAA TCC CA - 3′ | (XIVa) |
| 5′ - CCA AGC CTC CGA ATG CGA AA - 3′ | (XIVb). |

2. A method as claimed in claim 1 for detecting fungal infection of soil by pathogenic Pythium species, said method comprising:
obtaining a sample of soil; treating said sample to lyse fungal cells therein; using an oligonucleotide primer pair, effecting a polymerase chain reaction on DNA released by lysis of the fungal cells; and detecting DNA fragments generated by said polymerase chain reaction; wherein said primer pair comprises an 18- to 24-mer having the ability to hybridize to one of the oligonucleotide sequences of formulae Ia, Ib, IIa, IIb, IIIa, IIIb, IVa, IVb, Va, Vb, VIa, VIb, VIIa, VIIb, VIIIa, VIIIb, IXa, IXb, Xa and Xb:

| | |
|---|---|
| 5′ - TCA CTT GTG GGG TAA AGA AGA - 3′ | (Ia) |
| 5′ - AGA CCA CAA TAA AGC GGC - 3′ | (Ib) |
| 5′ - AGT CCC GCA CAC ACA CAT - 3′ | (IIa) |
| 5′ - ACT TCT CTC TTT GGG GAG TGG - 3′ | (IIb) |
| 5′ - TTC GTT CAG CCT CTG CAT - 3′ | (IIIa) |
| 5′ - TCG TTT CGG CTA TGA ATA CAG - 3′ | (IIIb) |
| 5′ - ACA AAT ATA CCA ACC ACA GCG - 3′ | (IVa) |
| 5′ - TTT GTA CTT GTG CAA TTG GC - 3′ | (IVb) |
| 5′ - AAC GAA TAT ACC AAC CGC TG - 3′ | (Va) |
| 5′ - TCA TCT ATT TGT GCA CTT CTT TTT - 3′ | (Vb) |
| 5′ - TCT TCT TTA CCC CAC AAG TGA - 3′ | (VIa) |
| 5′ - GCC GCT TTA TTG TGG TCT - 3′ | (VIb) |
| 5′ - ATG TGT GTG TGC GGG ACT - 3′ | (VIIa) |
| 5′ - CCA CTC CCC AAA GAG AGA AGT - 3′ | (VIIb) |
| 5′ - ATG CAG AGG CTG AAC GAA - 3′ | (VIIIa) |
| 5′ - CTG TAT TCA TAG CCG AAA CGA - 3′ | (VIIIb) |
| 5′ - CGC TGT GGT TGG TAT ATT TGT - 3′ | (IXa) |
| 5′ - GCC AAT TGC ACA AGT ACA AA -3′ | (IXb) |
| 5′ - CAG CGG TTG GTA TAT TCG TT - 3′ | (Xa) |
| 5′ - AAA AAG AAG TGC ACA AAT AGA TGA - 3′ | (Xb) |

3. A method as claimed in claim 1 for detecting fungal infection of soil or vegetables by pathogenic fungal species, said method comprising:
obtaining a sample of soil or vegetable; treating said sample to lyse fungal cells therein; using an oligonucleotide primer pair, effecting a polymerase chain reaction on DNA released by lysis of the fungal cells; and detecting DNA fragments generated by said polymerase chain reaction;
wherein said primer pair comprises an 18- to 24-mer having the ability to hybridize to one of the oligonucleotide sequences of formulae XIa, XIb, XIIa and XIIb, XIIIa, XIIIb, XIVa and XIVb:

| | |
|---|---|
| 5′ - GTT TGA ATG GAG TCC GAC CG - 3′ | (XIa) |
| 5′ - CGG CGT ACT TGC TTC GGA GC - 3′ | (XIb) |
| 5′ - TGG GAT TAA CGG GCA GAG AC - 3′ | (XIIa) |
| 5′ - TTT CGC ATT CGG AGG CTT GG - 3′ | (XIIb) |
| 5′ - CGG TCG GAC TCC ATT CAA AC - 3′ | (XIIIa) |
| 5′ - GCT CCG AAG CAA GTA CGC CG - 3′ | (XIIIb) |
| 5′ - GTC TCT GCC CGT TAA TCC CA - 3′ | (XIVa) |
| 5′ - CCA AGC CTC CGA ATG CGA AA - 3′ | (XIVb). |

4. A method as claimed in claim 2 wherein said primer pair comprises a pair of 18- to 24-mers having the ability to hybridize to a pair of the oligonucleotide sequences of formulae Ia and Ib or IIa and IIb or IIIa and IIIb or IVa and IVb or Va and Vb.

5. A method as claimed in claim 3 wherein said primer pair comprises a pair of 18- to 24-mers having the ability to hybridize to a pair of the oligonucleotide sequences of formulae XIa and XIb or XIIa and XIIb.

6. An assay method for detecting fungal infection of soil or vegetables by pathogenic fungal species, in particular M. acerina, F. carotae and Pythium species, said method comprising:
obtaining a sample of soil or vegetable; treating said sample to lyse fungal cells therein; using an oligonucleotide primer pair, effecting a polymerase chain reaction on DNA released by lysis of the fungal cells; contacting the DNA fragments generated by said polymerase chain reaction with a substrate having immobilized thereon a primer which comprises an 18- to 24-mer having the ability to hybridize to one of the oligonucleotide sequences of formulae Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIa, XIIb, XIIIa, XIIIb, XIVa and XIVb:

| | |
|---|---|
| 5′ - TCA CTT GTG GGG TAA AGA AGA - 3′ | (Ia) |
| 5′ - AGA CCA CAA TAA AGC GGC - 3′ | (Ib) |
| 5′ - AGT CCC GCA CAC ACA CAT - 3′ | (IIa) |
| 5′ - ACT TCT CTC TTT GGG GAG TGG - 3′ | (IIb) |
| 5′ - TTC GTT CAG CCT CTG CAT - 3′ | (IIIa) |
| 5′ - TCG TTT CGG CTA TGA ATA CAG - 3′ | (IIIb) |
| 5′ - ACA AAT ATA CCA ACC ACA GCG - 3′ | (IVa) |
| 5′ - TTT GTA CTT GTG CAA TTG GC - 3′ | (IVb) |
| 5′ - AAC GAA TAT ACC AAC CGC TG - 3′ | (Va) |
| 5′ - TCA TCT ATT TGT GCA CTT CTT TTT - 3′ | (Vb) |
| 5′ - TCT TCT TTA CCC CAC AAG TGA- 3′ | (VIa) |
| 5′ - GCC GCT TTA TTG TGG TCT- 3′ | (VIb) |
| 5′ - ATG TGT GTG TGC GGG ACT- 3′ | (VIIa) |
| 5′ - CCA CTC CCC AAA GAG AGA AGT- 3′ | (VIIb) |
| 5′ - ATG CAG AGG CTG AAC GAA- 3′ | (VIIIa) |
| 5′ - CTG TAT TCA TAG CCG AAA CGA- 3′ | (VIIIb) |
| 5′ - CGC TGT GGT TGG TAT ATT TGT- 3′ | (IXa) |
| 5′ - GCC AAT TGC ACA AGT ACA AA-3′ | (IXb) |
| 5′ - CAG CGG TTG GTA TAT TCG TT- 3′ | (Xa) |
| 5′ - AAA AAG AAG TGC ACA AAT AGA TGA - 3′ | (Xb) |
| 5′ - GTT TGA ATG GAG TCC GAC CG - 3′ | (XIa) |
| 5′ - CGG CGT ACT TGC TTC GGA GC - 3′ | (XIb) |
| 5′ - TGG GAT TAA CGG GCA GAG AC - 3′ | (XIIa) |
| 5′ - TTT CGC ATT CGG AGG CTT GG - 3′ | (XIIb) |
| 5′ - CGG TCG GAC TCC ATT CAA AC - 3′ | (XIIIa) |
| 5′ - GCT CCG AAG CAA GTA CGC CG - 3′ | (XIIIb) |
| 5′ - GTC TCT GCC CGT TAA TCC CA - 3′ | (XIVa) |
| 5′ - CCA AGC CTC CGA ATG CGA AA - 3′ | (XIVb); |

and detecting DNA fragments binding to said primer.

7. An 18- to 24-mer oligonucleotide primer hybridizable to an oligonucleotide sequence selected from those of formulae Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIA, XIIb, XIIIa, XIIIb, XIVa and XIVb.

8. A primer as claimed in claim 7 hybridizable to an oligonucleotide sequence selected from those of formulae Ia, Ib, IIa, IIb, IIIa, IIIb, IVa, IVb, Va, Vb, VIa, VIb, VIIa, VIIb, VIIIa, VIIIb, IXa, IXb, Xa and Xb.

9. A primer as claimed in claim 7 hybridizable to an oligonucleotide sequence selected from those of formulae XIa, XIb, XIIa, XIIb, XIIIa, XIIIb, XIVa and XIVb.

10. A primer as claimed in claim 7 wherein said primer comprises a sequence of formulae Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIa, XIIb, XIIIa, XIIIb, XIVa or XIVb or a derivative thereof.

11. A substrate having immobilized thereon at least one 18- to 24-mer oligonucleotide primer hybridizable to an oligonucleotide sequence selected from those of formulae Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIA, XIIb, XIIIa, XIIIb, XIVa and XIVb.

12. A substrate as claimed in claim 11 wherein said primer comprises a sequence of formulae Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIa, XIIb, XIIIa, XIIIb, XIVa or XIVb or a derivative thereof.

13. A primer composition comprising a pair of 18- to 24-mer oligonucleotide primers at least one of which is hybridizable to an oligonucleotide sequence of formula Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIa, XII, XIIIa, XIIIb, XIVa or XIVb optionally together with a carrier.

14. A primer composition as claimed in claim 13 wherein at least one of said pair is a primer comprising a sequence of formulae Ia, Ib, IIa, IIb, IIIa, IIIb, IVa, IVb, Va, Vb, VIa, VIb, VIIa, VIIb, VIIIa, VIIIb, IXa, IXb, Xa, Xb, XIa, XIb, XIIa, XIIb, XIIIa, XIIIb, XIVa or XIVb or a derivative thereof.

15. A composition as claimed in claim 13 comprising a pair of 18- to 24-mer oligonucleotide primers at least one of which is hybridizable to an oligonucleotide sequence of formula Ia, Ib, IIa, IIb, IIIa, IIIb, IVa, IVb, Va, Vb, VIa, VIb, VIIa, VIIb, VIIIa, VIIIb, IXa, IXb, Xa or Xb.

16. A composition as claimed in claim 13 comprising a pair of 18- to 24-mer oligonucleotide primers at least one of which is hybridizable to an oligonucleotide sequence of formulae XIa, XIb, XIIa, XIIb, XIIIa, XIIIb, XIVa or XIVb.

17. A kit for the performance of the assay method of any one of claims 1 to 5, said kit comprising at least one primer pair as defined in any one of claims 1 to 5 together with instructions for the performance of the assay method.

18. A process for the extraction of nucleic acid from soil which process comprises:
1) contact a sample of about 0.1 to 1 g, preferably about 0.5 g, soil taken from a mixed sample of at least 100 g, preferably at least 200 g, soil with a fungal cell lysing agent;
2) centrifuge at least 10000×g for at least 10 minutes and collect the supernatant;
3) contact the supernatant with a particulate DNA-binding agent;
4) centrifuge and collect the DNA-bearing particulate;
5) suspend the particulate in an aqueous solution of a chaotropic agent (e.g. aqueous guanidine thiocyanate solution), centrifuge and collect the DNA-bearing particulate;
6) repeat step (5) at least once;
7) suspend the particulate in aqueous salt/ethanol wash solution, centrifuge and collect the DNA-bearing particulate;
8) repeat step (7) at least once;
9) suspend the particulate in an aqueous solution of a DNA-release agent;
10) centrifuge and collect the DNA-containing supernatant; and optionally
11) resuspend the particulate in an aqueous solution of a DNA-release agent, centrifuge and collect and combine the supernatant.

19. A kit for nucleic acid extraction from soil, which kit comprises:
i) an aqueous fungal cell lysing agent;
ii) a DNA-binding particulate;
iii) an aqueous solution of a chaotropic agent (e.g. guanidine thiocyanate);
iv) an aqueous solution of salt and ethanol; and
v) an aqueous solution of a DNA-release agent;
together with instructions for the use of said kit in the process of claim 13.

20. A process for the extraction of pathogen DNA from host vegetable tissue, which process comprises:
i) contact at least 20 mg of dry powdered plant tissue (preferably surface tissue such as peel) with at least 5 μL/mg dry tissue of an aqueous fungal cell lysing agent;
ii) incubate;
iii) mix with at least 4.5 μL/mg dry tissue of an aqueous solution of a protein and polysaccharide precipitating agent;
iv) centrifuge and collect DNA-containing supernatant;
v) filter;
vi) contact DNA-containing filtrate with a DNA-binding substrate and centrifuge;
vii) wash the DNA-carrying substrate with an aqueous ethanolic solution, centrifuge and remove the liquid phase;
viii) repeat step (vii) at least once;
ix) dry the DNA-carrying substrate; and
x) contact the substrate with an aqueous solution of a DNA release agent, centrifuge and collect the DNA-containing supernatant.

21. A kit for pathogen DNA extraction from host vegetable tissue, which kit comprises:
a) a fungal cell lysing agent;
b) an aqueous solution of a protein and polysaccharide precipitating solution;
c) a DNA-binding substrate;
d) an aqueous ethanolic wash solution; and
e) an aqueous solution of a DNA release agent;
together with instructions for the use of said kit for pathogen DNA extraction from host vegetable tissue.


SEQUENCE LISTING (33 sequences)

SEQ ID NO: 1 - DNA, 21 bp, Artificial Sequence
  Chemically-synthesized oligonucleotide primer
tcacttgtggggtaaagaaga

SEQ ID NO: 2 - DNA, 18 bp, Artificial Sequence
  Chemically-synthesized oligonucleotide primer
agaccacaataaagcggc

SEQ ID NO: 3 - DNA, 18 bp, Artificial Sequence
  Chemically-synthesized oligonucleotide primer
agtcccgcacacacacat

SEQ ID NO: 4 - DNA, 21 bp, Artificial Sequence
  Chemically-synthesized oligonucleotide primer
acttctctctttggggagtgg

SEQ ID NO: 5 - DNA, 18 bp, Artificial Sequence
  Chemically-synthesized oligonucleotide primer
ttcgttcagcctctgcat

SEQ ID NO: 6 - DNA, 21 bp, Artificial Sequence
  Chemically-synthesized oligonucleotide primer
tcgtttcggctatgaatacag

SEQ ID NO: 7 - DNA, 21 bp, Artificial Sequence
  Chemically-synthesized oligonucleotide primer
acaaatataccaaccacagcg

SEQ ID NO: 8 - DNA, 20 bp, Artificial Sequence
  Chemically-synthesized oligonucleotide primer
tttgtacttgtgcaattggc

SEQ ID NO: 9 - DNA, 20 bp, Artificial Sequence
  Chemically-synthesized oligonucleotide primer
aacgaatataccaaccgctg

SEQ ID NO: 10 - DNA, 24 bp, Artificial Sequence
  Chemically-synthesized oligonucleotide primer
tcatctatttgtgcacttcttttt

SEQ ID NO: 11 - DNA, 21 bp, Artificial Sequence
  Chemically-synthesized oligonucleotide primer
tcttctttaccccacaagtga

SEQ ID NO: 12 - DNA, 18 bp, Artificial Sequence
  Chemically-synthesized oligonucleotide primer
gccgctttattgtggtct

SEQ ID NO: 13 - DNA, 18 bp, Artificial Sequence
  Chemically-synthesized oligonucleotide primer
atgtgtgtgtgcgggact

SEQ ID NO: 14 - DNA, 21 bp, Artificial Sequence
  Chemically-synthesized oligonucleotide primer
ccactccccaaagagagaagt

SEQ ID NO: 15 - DNA, 18 bp, Artificial Sequence
  Chemically-synsthesized oligonucleotide primer
atgcagaggctgaacgaa

SEQ ID NO: 16 - DNA, 21 bp, Artificial Sequence
  Chemically-synthesized oligonucleotide primer
ctgtattcatagccgaaacga

SEQ ID NO: 17 - DNA, 21 bp, Artificial Sequence
  Chemically-synthesized oligonucleotide primer
cgctgtggttggtatatttgt

SEQ ID NO: 18 - DNA, 20 bp, Artificial Sequence
  Chemically-synthesized oligonucleotide primer
gccaattgcacaagtacaaa

SEQ ID NO: 19 - DNA, 20 bp, Artificial Sequence
  Chemically-synthesized oligonucleotide primer
cagcggttggtatattcgtt

SEQ ID NO: 20 - DNA, 24 bp, Artificial Sequence
  Chemically-synthesized oligonucleotide primer
aaaaagaagtgcacaaatagatga

SEQ ID NO: 21 - DNA, 20 bp, Artificial Sequence
  Chemically-synthesized oligonucleotide primer
gtttgaatggagtccgaccg

SEQ ID NO: 22 - DNA, 20 bp, Artificial Sequence
  Chemically-synthesized oligonucleotide primer
cggcgtacttgcttcggagc

SEQ ID NO: 23 - DNA, 20 bp, Artificial Sequence
  Chemically-synthesized oligonucleotide primer
tgggattaacgggcagagac

SEQ ID NO: 24 - DNA, 20 bp, Artificial Sequence
  Chemically-synthesized oligonucleotide primer
tttcgcattcggaggcttgg

SEQ ID NO: 25 - DNA, 20 bp, Artificial Sequence
  Chemically-synthesized oligonucleotide primer
cggtcggactccattcaaac

SEQ ID NO: 26 - DNA, 20 bp, Artificial Sequence
  Chemically-synthesized oligonucleotide primer
gctccgaagcaagtacgccg

SEQ ID NO: 27 - DNA, 20 bp, Artificial Sequence
  Chemically-synthesized oligonucleotide primer
gtctctgcccgttaatccca

SEQ ID NO: 28 - DNA, 20 bp, Artificial Sequence
  Chemically-synthesized oligonucleotide primer
ccaagcctccgaatgcgaaa

SEQ ID NO: 29 - DNA, 19 bp, Artificial Sequence
  Chemically-synthesized oligonucleotide primer
tccgtaggtgaacctgcgg

SEQ ID NO: 30 - DNA, 20 bp, Artificial Sequence
  Chemically-synthesized oligonucleotide primer
gctgcgttcttcatcgatgc

SEQ ID NO: 31 - DNA, 20 bp, Artificial Sequence
  Chemically-synthesized oligonucleotide primer
gcatcgatgaagaacgcagc

SEQ ID NO: 32 - DNA, 20 bp, Artificial Sequence
  Chemically-synthesized oligonucleotide primer
tcctccgcttattgatatgc

SEQ ID NO: 33 - DNA, 22 bp, Artificial Sequence
  Chemically-synthesized oligonucleotide primer
ggaagtaaaagtcgtaacaagg